(19) United States
(12) Patent Application Publication
(10) Pub. No.: US 2023/0039403 A1
(43) Pub. Date: Feb. 9, 2023

(54) System for detecting fungus, virus, and disease-causing pathogens in an agricultural industry using artificial intelligence.

(71) Applicant: Keith Louis De Santo, St Petersburg, FL (US)

(72) Inventor: Keith Louis De Santo, St Petersburg, FL (US)

(21) Appl. No.: 17/879,932

(22) Filed: Aug. 3, 2022

Related U.S. Application Data
(63) Continuation-in-part of application No. 16/828,832, filed on Mar. 24, 2020.

(51) Int. Cl. G01N 33/24; G01P 5/00; G05D 1/00; A01M 7/00; B64C 39/02; B64D 1/18

(52) U.S. Cl. CPC G01N 33/24; A01M 7/005; B64C 39/024; B64D 1/18; G01P 5/00; G05D 1/0016; B64C 2201/122; B64C 2201/127; G01N 2033/245

(57) ABSTRACT

A system for detecting fungus, virus, and disease-causing pathogens in an agricultural industry using artificial intelligence, which comprises: an unmanned aerial vehicle (UAV); and a ground terminal telecommunicating with the UAV using a wireless access communication, wherein the UAV comprises a wireless transmitter for transmitting data to and from the ground terminal, an array of cantilevers on a substrate located at one side of the wireless transmitter, a blacklight located at the other side of the wireless transmitter, and a sensory part located on the wireless transmitter, wherein the cantilever is made up of beams anchored at one end and projecting into space, and wherein the sensory part comprises a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor, and wherein the ground terminal comprises an artificial intelligence machine-learning and data-mining platform wirelessly telecommunicating with the UAV.

### REFERENCE TO RELATED APPLICATION

This application is a continuation-in-part of and claims the benefit of and priority to U.S. Pat. Application Serial No. 16/828,832, filed on Mar. 24, 2020 entitled "Mobile AI, Cantilever, Robot and Drone Applications" (pending), the entire contents of which are incorporated herein by reference.

### FIELD OF THE INVENTION

The present disclosure relates to a system for detecting fungus, virus, and disease-causing pathogens in an agricultural industry using artificial intelligence.

### BACKGROUND OF THE INVENTION

Unfortunately, a fungus, a disease and virus-causing pathogens are often present and cause issues in an agricultural industry. Agriculture is the science, art, or practice of cultivating soil, producing plants' crops, and raising livestock, and, in varying degrees, agriculture includes preparation and marketing of resulting products. Agriculture is sensitive to environmental degradation, such as biodiversity loss, desertification, soil degradation and global warming, all of which can cause decreases in crop yield. Major agricultural products can be broadly grouped into foods, fibers, fuels, and raw materials.

A fungus is any member of a group of eukaryotic organisms that includes microorganisms, such as yeasts and molds. A disease is a particular abnormal condition that negatively affects a structure or a function of all or a part of an organism. A pathogen is any organism that can produce disease. As a result, pathogens are often also referred to as infectious agents or germs.

There is currently an unmet need to detect fungus, diseases, and virus-causing pathogens in the agricultural industry in order to produce safe foods, fibers, fuels, and raw materials. Pathogens in agriculture are currently detected by other applications that require time, higher costs, and expensive technology.

### SUMMARY OF THE INVENTION

An aspect of the present disclosure is to provide a system for detecting fungus, virus, and disease-causing pathogens in an agricultural industry using artificial intelligence, which comprises an unmanned aerial vehicle (UAV) and a ground terminal telecommunicating with the UAV using a wireless access communication, wherein the UAV comprises a wireless transmitter for transmitting data to and from the ground terminal, an array of cantilevers on a substrate located at one side of the wireless transmitter, a blacklight located at the other side of the wireless transmitter, and a sensory part located on the wireless transmitter, wherein the cantilever is made up of beams anchored at one end and projecting into space, and wherein the sensory part comprises a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor, and wherein the ground terminal comprises an artificial intelligence machine-learning and data-mining platform wirelessly telecommunicating with the UAV.

The high-definition microscope camera may have at least one lens ranging from 100 times to 100,000,000 times magnification.

The cantilevers may be thin wire nano-beams made of glass, silicone, or metal.

The cantilevers may be one or more and 10 million or less on a substrate.

The cantilevers may capture, weigh, and detect pathogens on a femtogram (10⁻¹⁵) or yoctogram (10⁻²⁴) scale.

The cantilevers may be configured to calculate wind speed.

The UAV may further include a soil collector for transmitting soil data to the artificial intelligence machine-learning and data-mining platform.

The UAV may further include a rhamnolipid biosurfactants applicator for eliminating the pathogens by spraying rhamnolipid biosurfactants to agricultural products.

The artificial intelligence machine-learning and data-mining platform may comprise mapping software, which can map out an area to determine whether one or more fungus, virus, and disease-causing pathogens are located within the area.

The mapping software may form a three-dimensional schematic view with x, y, and z data points.

The artificial intelligence machine-learning and data-mining platform may further include a fungus, virus, and disease-causing pathogens detection mechanism for determining a type of the pathogens, determining which one of the pathogens can cause diseases, and determining how to negate the pathogens.

The system may further include a ground-moving robot using wireless access communication with a ground terminal, which may include a wireless transmitter for transmitting data to and from the ground terminal, an array of cantilevers on a substrate located at one side of the wireless transmitter, a blacklight located at the other side of the wireless transmitter, a sensory part located on the wireless transmitter, wherein the sensory part comprises a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor, and a mechanical arm located in front of the high-definition microscope camera or on the array of cantilevers for collecting a ground soil and/or removing one or more fungus, virus, or disease-causing pathogens, wherein the cantilever is made up of beams anchored at one end and projecting into space.

The mechanical arm may range from 10 microns to 3 centimeters in length.

### BRIEF DESCRIPTION OF DRAWINGS

It should be noted that the shapes and sizes of the elements in the drawings are not to scale and are merely intended to illustrate the invention, in which:

FIG. 1A shows an isometric schematic view of an unmanned aerial vehicle (UAV) or a drone according to one embodiment of the present disclosure; FIG. 1B shows a top view of an unmanned aerial vehicle (UAV) or a drone according to one embodiment of the present disclosure; and FIG. 1C shows a front view of an unmanned aerial vehicle (UAV) or a drone according to one embodiment of the present disclosure.

FIG. 2 shows a schematic view of a cantilever having a beam anchored at one end on a substrate and projecting into space according to one embodiment of the present disclosure.

FIG. 3A shows an isometric schematic view of a ground-moving robot having a mechanical arm according to one embodiment of the present disclosure; and FIG. 3B shows a side view of a ground-moving robot having a mechanical arm according to one embodiment of the present disclosure.

FIG. 4 shows a schematic diagram illustrating a wireless access communication between an unmanned aerial vehicle (UAV) or a drone, or a ground-moving robot and a ground terminal.

### DETAILED DESCRIPTION OF THE INVENTION

Our new artificial intelligence (AI) platforms are based on Mobile Cantilever nanotechnology, data mining through high-definition pictures and machine learning algorithms known as "MCology™". The AI platform learns about plant and crop pathogens, how to detect them and how to negate pathogens on a single plant or an entire crop. Our new invention has begun to anticipate and forecast threats to plants, bushes, trees, and entire crops.

Our new disease, virus, fungus, contamination, air quality/pollution level detection process uses numerous algorithms. Cantilevers can calculate the weight of pathogens in femtograms (10⁻¹⁵ grams), and determine the level of air pollution while our AI platform can determine the mass of pathogens, contamination type, and what particles are in the air. Our new MCology™ platform can detect many different points of data such as what toxin or pathogen is affecting plants, trees, bushes, or entire crops. MCology™ detects fungus, diseases, viruses, and contamination type, particular molecules, the stage of the pathogen, and how to control it using our AI, data mining and machine learning algorithms. If a certain bacterium is causing disease, we can determine which bacteria it is. This new invention can detect how long the bacteria has been affecting the plant by the level of colonies and size of the bacterium. This new invention can also at times determine where the bacteria came from such as waste water run-off, pests, agricultural machinery, air pollution, or weather events. The new invention can also determine how long the issue has been affecting the plant by algorithms that calculate and learn the duration of the issue using machine learning. Each time an event has occurred, the AI platform learns from that event.

Daily rapid sampling using our invention can also predict what kind of outcome will occur in the field of agricultural crops.

The present invention can measure the weight and size of biological and or chemical elements by using an unmanned drone, robot, land, air or sea roving mobile vehicle equipped with an array of cantilevers that relay data to an AI platform. The drones and robots can be powered by solar energy, nuclear energy, battery or combustion engine and are unmanned. The vehicles can be operated by wireless technology, cell phones, or via satellite. The size and weight of the unmanned vehicles range from large multi-ton to nanotechnology-sized vehicles. With this new invention, we can detect the signature of pathogens with ease. With our new detection system, we are able to immediately ascertain the type of pathogen, particular molecules, the stage of the pathogen and how to control it using AI data mining.

Disease in plants, food, and water including the spreading of pathogens from environmental contamination contact can be controlled if an alert system is employed and an assessment is made immediately. Our application is just that system. Our instantaneous rapid detection of virulence can be completed every minute, every day, or every month. Our platform can be used for agricultural applications indoors and outdoors. MCology™ is also an early warning system of pathogens in the food, water, and agricultural industries before they are sold on the store shelves.

### Cantilever Technology

Cantilevers utilizing nanotechnology have been in use since early 2000. A cantilever can be described as a small diving board. The board is anchored (anchored end) at one end and free to fluctuate at the other end (non-anchored end). Cantilevers can be perpendicular to a flat, vertical or slanted surface and are rigid structures. They may be made of any structural element such as a thin wire made out of silicone. If the cantilever is subjected to a load of any type of weight, it will bend. The stress at the non-anchored end from the load can be weighed by the angle of the board using different lengths. Our mobile cantilever technology utilizes stacked arrays of many cantilevers made of glass, silicone, and metal.

Current cantilever technology uses static applications that are limited in process. These applications take time to retrieve the data from the cantilevers and then proceed with analyzing the data that may be inaccurate. This procedure is time-consuming and costly. Rapid detection and analysis from our mobile drones and robots can produce data immediately. Our Mobile Cantilever Technology can detect the full array of pathogens using AI algorithms immediately.

One component of our application of pathogen and molecular detection is done by size. Our technology can detect sizes of particles in femtograms. A femtogram is a measurement of weight where a femtogram is equal to 10⁻¹⁵ grams.

Our AI system is able to utilize data-mining collected from the cantilevers by learning the structure, weight, mass size, behavior, fingerprints and footprints of pathogenic material.

Robots, drones and cantilevers through Artificial Intelligence "AI" platforms are replacing workers with new technology and machine learning algorithms. Indoor environments are benefiting from this new technology by learning about indoor environments, their cleanliness, the causes of unsanitary conditions and the sanitizing of the indoor space. Monitoring an indoor environment such as a nursery costly and time-consuming. Our application is quick, cost efficient and can be deployed with ease.

Natural intelligence is demonstrated by humans and animals. Artificial Intelligence "AI" also referred to as "machine intelligence" is non-human intelligence exhibited by machines. AI machines are also called intelligent agents. Machine Learning is the study and implementation of algorithms and statistical models that computers "learn" from. The computers set out to use data mining (pattern discovery) to perform specific tasks. The learning can be in the form of patterns, interpretations and presumptions using data mining to arrive at conclusions.

High-definition pictures, video, statistics among other data are also used to extract information from the complied data to detect levels of toxins and pathogens and forecast future events. For the sake of this invention, Artificial Intelligence, machine learning and data mining should be considered working together to form our AI platform.

A robot is defined as a machine that is programmable and can carry out a series of tasks. A robot that is programmed and managed by an AI machine learning machine platform is a "Robot Learning Machine." Our Robots have cantilevers on or within the body of the robot.

An unmanned aerial vehicle (UAV) or a drone is defined as a pilotless flying robot. Our drones have cantilevers on or within the body of the drone.

For a better understanding of the contents, features, and effects of the present disclosure, the following embodiments are given as examples with reference to the drawings.

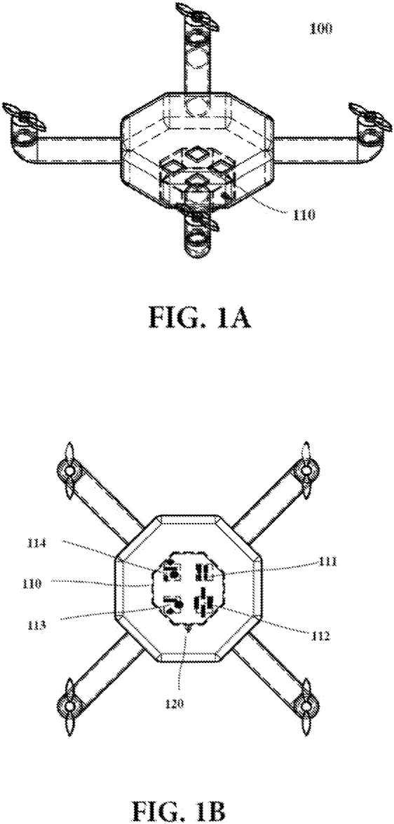

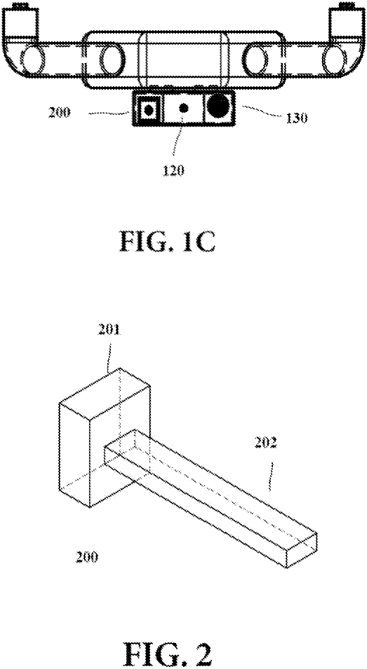

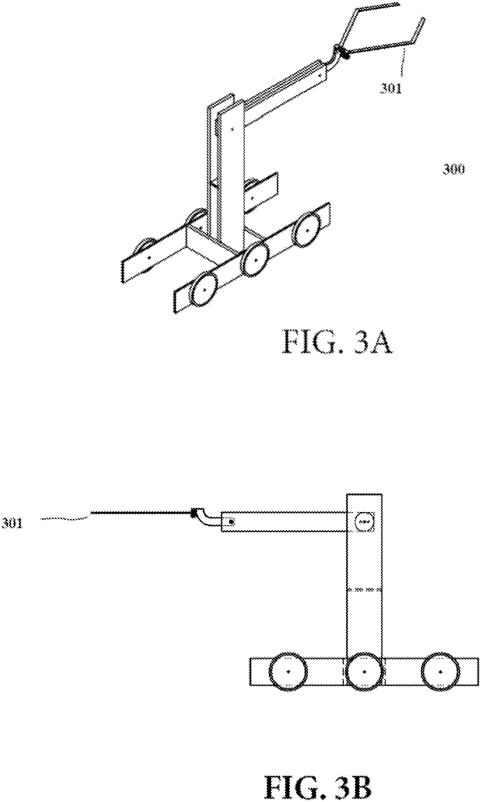

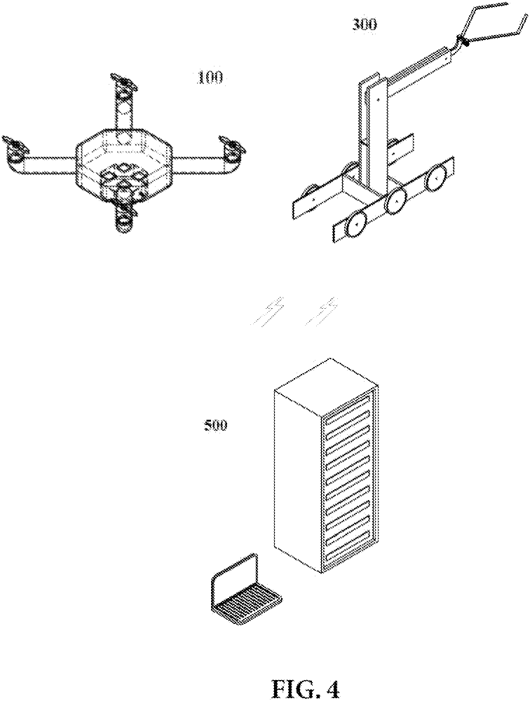

FIGS. 1A to 1C show a schematic view of an unmanned aerial vehicle (UAV) or a drone 100 according to one embodiment of the present disclosure. FIG. 2 shows a schematic view of a cantilever 200 having a beam anchored at one end on a substrate and projecting into space. FIGS. 3A and 3B show an isometric schematic view of a ground-moving robot 300. FIG. 4 shows a schematic diagram illustrating a wireless access communication between the UAV or drone 100, or the robot 300 and a ground terminal 500.

The UAV or drone 100 detects fungus, virus, and disease-causing pathogens in the agricultural industry. The UAV or drone 100 communicates with the ground terminal 500 having one or more artificial intelligence platforms. Each of one or more artificial intelligence platforms utilizes data mining and machine learning algorithms to control a UAV or a drone 100 and a robot 300. The UAV or drone 100, or the robot 300 operates in the air or in a body of water, or on land. Embedded in the robot 300 or the UAV or drone 100 is one or more cantilevers 200 to detect fungus, disease and/or viruses in soil or to detect contamination or air pollution particles. The cantilevers capture, weigh, and detect pathogens on a femtogram (10⁻¹⁵) or yoctogram (10⁻²⁴) scale. The data mining and machine learning algorithms retrieve data from one or more cantilevers 200 through a wireless transmitter 120 and analyze the weight of pathogens in femtograms by way of examining high-definition microscope pictures.

The UAV or drone 100 includes a blacklight 130 located at one side of the wireless transmitter 120, and a sensory part 110 located on the wireless transmitter 120. The sensory part 110 may be divided and sorted into four categories or parts (111, 112, 113, 114), if necessary, and include, but is not limited to, a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor. The camera can take one or a plurality of high-definition analytical photographs of a leaf, a branch, a stem of a plant, a tree, a bush, or a flower with at least one lens ranging 100 times through 100,000,000 times magnification. With these sensors, a structure, a weight, a mass size, one or a plurality of behaviors, and one or a plurality of prints of fungus, disease and virus-causing pathogens may be determined and reported to the artificial intelligence platforms.

Referring to FIGS. 3A and 3B, the ground-moving robot 300 uses wireless access communication with the ground terminal 500, wherein the ground-moving robot 300 comprises a wireless transmitter for transmitting data to and from the ground terminal 500, an array of cantilevers 200 on a substrate located at one side of the wireless transmitter, a blacklight located at the other side of the wireless transmitter, a sensory part located on the wireless transmitter, wherein the sensory part comprises a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor, and a mechanical arm 301 located in front of the high-definition microscope camera or on the array of cantilevers for collecting a ground soil and/or removing one or more fungus, virus, or disease-causing pathogens, wherein the cantilever is made up of beams anchored at one end and projecting into space. The mechanical arm 301 may collect soil or pathogens samples for research and identify them. The mechanical arm 301 may range from 10 microns to 3 centimeters in length. The mechanical arm 301 extends from a beam that pivots from a mechanical pivot device affixed to a table or a conveyor belt and collects ground soil and/or removes one or more fungus, virus, or disease-causing pathogens. The mechanical arm 301 may move up and down through a mechanical pivot device. The mechanical arm 301 can have mechanical hands for opening and closing on microscope slides.

FIG. 4 shows one exemplary embodiment of the wireless access communication between the UAV or drone 100 and the ground terminal 500. The system for surveying one or a plurality of products in the agricultural industry can determine one or a plurality of pathogens. The ground terminal 500 comprises an artificial intelligence platform to compile data associated with the agricultural industry. The artificial intelligence platform maps out an area associated with the agricultural industry by scanning a perimeter of the area using the UAV or drone 100. The artificial intelligence platform detects whether the area is enclosed with an upright barrier structure placed around a plurality of sides of the area. The artificial intelligence platform maps out the area to determine whether there is one or a plurality of internal items located within the area. The system further includes a bacteria, fungus, virus, and disease-causing pathogens detection mechanism to determine which one of the one or the plurality of pathogens are causing one or a plurality of diseases. The bacteria, fungus, virus, and disease-causing pathogens detection mechanism is further configured to determine a type of the one or the plurality of the pathogens. The bacteria, fungus, virus, and disease-causing pathogens detection mechanism is also configured to determine one or a plurality of molecules associated with one or the plurality of the pathogens. The bacteria, fungus, virus, and disease-causing pathogens detection mechanism is further configured to determine how to control one or the plurality of pathogens using artificial intelligence data mining generated by the artificial intelligence platform.

The ground terminal 500 comprises a learning platform to provide step-by-step instructions to the UAV or drone 100 or the robot 300 in order to guide the UAV or drone 100 or the robot 300 to be affixed to a still structure. The ground terminal further includes a calculation software platform to determine a weight associated with a branch of the still structure. The ground terminal 500 also comprises an affixing mechanism to affix the UAV or drone 100 or the robot 300 to the still structure.

### Example 1

Drones, cantilevers and AI in the agricultural industry. One of the biggest challenges in all agricultural industries including the hemp and cannabis industries is to contain and avoid disease, fungus and viruses. An early warning system and information about how to correct or avoid the threat will not only save money from crop destruction but our new system may save businesses from going under.

An example would be that our system would learn what caused destruction at specific times with specific crops such as in the cannabis industry. At a grove in California, most of the cannabis plants would not flower and no one knew why. Our system learned that a cannabis canker was the culprit long before the problem was known. Our system also learned how to manage the problem by learning when to apply agricultural products such as rhamnolipid biosurfactants, how much was needed and what ratio of mono to di-rhamnolipid biosurfactants was effective. Our sensors are both active and passive and are divided up into 4 categories.

### Example 2

Robots, cantilevers and AI in the agricultural industry. In order to take high definition pictures for our data mining algorithms, drones must be very still in order to get 360 degrees and avoid blurry pictures. Our robots can affix themselves to the walls and grounds or erect a quick platform where wind and or bright light is either needed or avoided depending on what the machine learning algorithm has learned. An example of this is during the rainy season where wind and rain pose a problem for cantilever reading particles as well as obtaining high-definition microscope pictures. The quick setup of a mobile enclosure negates these problems. The AI platform would manage all aspects of deploying such a cover through the robot. A simple example of our enclosure would be similar to a spring-loaded tent that is big enough to cover a branch or a plant and shield it from weather elements that can just as easily be deconstructed in seconds with very little effort on behalf of the AI platform.

### Example 3

Our learning platform has also learned how drones can affix themselves to plants and trees or just rest on single branches of trees or several thin branches to gather data. Algorithmic calculations determine the weight the branch or leaf can withstand by measuring the thickness of branch or cluster of leaves by drone mapping. An example of this would be if is noon and in the middle of the summer where the temperature is over 80 degrees and sun is directly overhead. When working with trees such as the African palm tree that can grow over 20 meters, the drone would not only need shade to take some high-definition pictures underneath the palm leaf but need to sample the surrounding air. Resting on a leaf with a shade above and enough drag coefficient on the surface of the leaf to not get blown about by a gust of wind. Cantilevers can be programmed to calculate wind speed. The AI platform not only learns from its prior mistakes but also learns to take less and less time to complete each task. In one instance, our machine learning algorithm was able to not set the drone down because a large snake was occupying the branch.

### Example 4

One of our drones' purposes in the agricultural industry is to survey and calculate problematic areas and determine sanitary or unsanitary conditions of an indoor or outdoor, open or closed structure housing agricultural products. Our drones compile the following data. They map out the area of the grove, nursery or farm that needs to be scanned. If the structure is closed like that of a nursery, the AI platform directs the drones and robots to work together to map out the internal items that are located inside the structure by using specific algorithms. The combined data of structure area and internal contents will form the "shell" and be transferred into the AI platform. Then, a mapping software of the indoor structure will use visual, laser mapping, and grid calculation software whereas the entire area including the internal contents of the structure will form a three-dimensional schematic view with x, y, and z data points. This data will be calculated and transferred into our AI platform. Patterns of soil collection and its moisture will be transferred into the AI platform to see if the soil collection is arid or not and to what degree the aridity is. The irrigation system in the crop area can be revised to add or limit water. Our AI platform can calculate a change in water distribution flow or a problem with the entire irrigation system. An example of this would be that after building a database of irrigation successes and failures, data mining has learned about other facilities' various irrigation systems and which ones had more success in utilizing less water.

### Example 5

Gathering data without tainting the data. Using Robots and Drones will limit errors that humans are prone to making. Humans tend to transfer pathogens on their clothes and the soles of shoes. These infection metrics can be negated. They also tend to make mistakes in harvest calculations. Using AI to map the area of the grove will also improve the quality and quantity of the crop harvest. An example of this is by using an AI algorithm to pre-determine if the crop is viable, valuable and can be easily harvested. By this, high-definition pictures through data mining, such as footprints and fingerprints of healthy cell structures build learning from past harvests. Through this, our platform can forecast some crop events such as what level of health is the plant at the time of harvest.

### Example 6

Our AI platform of algorithms learns about certain hazards such as over and under fertilizing, over and under watering and certain weather events that are either positive or have a negative effect. An example would be when a toxic chemical is emitted into the air or soil by accident, our cantilevers will detect immediately what the toxin is and where the problem started. Our AI platform learns how to correct those problems.

### Example 7

During many agricultural applications, pathogens can be introduced into greenhouses, nurseries and outdoor crops through machinery and tools used in the fields. Our AI algorithm scans and tests for toxins and pathogens before the item is used in the field or on crops. Machine learning learns how to avoid these problems by understanding how and when a toxin or pathogen is introduced to the plant.

1. A system for detecting fungus, virus, and disease-causing pathogens in an agricultural industry using artificial intelligence, which comprises:
an unmanned aerial vehicle (UAV); and
a ground terminal telecommunicating with the UAV using a wireless access communication, wherein
the UAV comprises a wireless transmitter for transmitting data to and from the ground terminal, an array of cantilevers on a substrate located at one side of the wireless transmitter, a blacklight located at the other side of the wireless transmitter, and a sensory part located on the wireless transmitter, wherein the cantilever is made up of beams anchored at one end and projecting into space, and wherein the sensory part comprises a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor, and wherein
the ground terminal comprises an artificial intelligence machine-learning and data-mining platform wirelessly telecommunicating with the UAV.

2. The system of claim 1, wherein the high-definition microscope camera has at least one lens ranging from 100 times to 100,000,000 times magnification.

3. The system of claim 1, wherein the cantilevers are thin wire nano-beams made of glass, silicone, or metal.

4. The system of claim 1, wherein the cantilevers are one or more and 10 million or less on a substrate.

5. The system of claim 1, wherein the cantilevers capture, weigh, and detect pathogens on a femtogram (10⁻¹⁵) or yoctogram (10⁻²⁴) scale.

6. The system of claim 1, wherein the cantilevers are configured to calculate wind speed.

7. The system of claim 1, wherein the UAV further comprises a soil collector for transmitting soil data to the artificial intelligence machine-learning and data-mining platform.

8. The system of claims 1, wherein the UAV further comprises a rhamnolipid biosurfactants applicator for eliminating the pathogens by spraying rhamnolipid biosurfactants to agricultural products.

9. The system of claim 1, wherein the artificial intelligence machine-learning and data-mining platform comprises mapping software, which can map out an area to determine whether one or more fungus, virus, and disease-causing pathogens are located within the area.

10. The system of claim 9, wherein the mapping software forms a three-dimensional schematic view with x, y, and z data points.

11. The system of claim 1, wherein the artificial intelligence machine-learning and data-mining platform further comprises a fungus, virus, and disease-causing pathogens detection mechanism for determining a type of the pathogens, determining which one of the pathogens can cause diseases, and determining how to negate the pathogens.

12. The system of claim 1, wherein the artificial intelligence machine-learning and data-mining platform is MCology™ platform.

13. The system of claim 1, further comprising a ground-moving robot using wireless access communication with the ground terminal, wherein the ground-moving robot comprises a wireless transmitter for transmitting data to and from the ground terminal, an array of cantilevers on a substrate located at one side of the wireless transmitter, a blacklight located at the other side of the wireless transmitter, a sensory part located on the wireless transmitter, wherein the sensory part comprises a scanner with a high-definition microscope camera, a laser sensor for three-dimensional areal mapping, an infrared sensor, a humidity sensor, a thermostat, a gas sensor, a thermal sensor, an optical dust particle sensor, an electro-optical sensor, and an air quality sensor, and a mechanical arm located in front of the high-definition microscope camera or on the array of cantilevers for collecting a ground soil and/or removing one or more fungus, virus, or disease-causing pathogens, wherein the cantilever is made up of beams anchored at one end and projecting into space.

14. The system of claim 13, wherein the mechanical arm ranges from 10 microns to 3 centimeters in length.
